(19) United States
(12) Patent Application Publication
(10) Pub. No.: US 2021/0121403 A1
(43) Pub. Date: Apr. 29, 2021

(54) METHODS OF PREPARING FREE-FLOWING POWDER FROM CANNABINOID OILS

(71) Applicant: Nano Pharmaceutical Laboratories, LLC, Denver, CO (US)

(72) Inventors: Robert Niichel, Greenwood Village, CO (US); Dan A. Finkbeiner, Erie, CO (US); Clayton J. Nelsen, Aurora, CO (US)

(21) Appl. No.: 16/773,833

(22) Filed: Jan. 27, 2020

Related U.S. Application Data
(60) Provisional application No. 62/926,342, filed on Oct. 25, 2019.

(51) Int. Cl. A61K 9/14; A61K 31/05; A61K 47/38; A61K 47/02; A61K 47/44; A61K 47/46; A61K 47/22; A61K 47/12; A61K 47/18; A61K 47/10

(52) U.S. Cl. CPC A61K 9/14; A61K 31/05; A61K 47/38; A61K 47/02; A61K 47/10; A61K 47/46; A61K 47/22; A61K 47/12; A61K 47/18; A61K 47/44

(57) ABSTRACT

Methods of preparing free-flowing powder from cannabinoid oils, including, but not limited to broad or full spectrum cannabidiol (CBD) oil, generally include granulating a cannabinoid oil with a granulating agent to form a granulated mixture, mixing a binder and/or filler with the granulated mixture to adjust the size of the granules in the granulated mixture, and mixing flow agent with the granulated mixture. The methods described herein can also include mixing bioavailability enhancer with the granulated mixture to improve the bioavailability of the cannabinoid compound in the free-flowing powder.

### CROSS-REFERENCE TO RELATED APPLICATION(S)

This non-provisional patent application claims the benefit of priority to U.S. Provisional Patent Application No. 62/926,342, titled METHOD OF PREPARING FREE-FLOWING CANNABIDIOL POWDER, and filed Oct. 25, 2019, which is incorporated by reference herein in its entirety by reference thereto.

### TECHNICAL FIELD

This disclosure relates to methods of preparing free-flowing powder from cannabinoid oils, including, but not limited to broad or full spectrum cannabidiol (CBD) oil, cannabigerol (CBG) oil, cannibinol (CBN) oil, tetrahydrocannabinoil (THC) oil, and tetrahydrocannabivarin (THCV). Also described herein are methods for adding bio-enhancers to the free-flowing powder as part of the conversion from cannabinoid oils to powders to thereby increase the bioavailability of the cannabinoid compound.

### BACKGROUND

Cannabinoids are a class of diverse chemical compounds that act on cannabinoid receptors. Cannabinoid receptors are located throughout the body of some animals and form part of the endocannabinoid system. The endocannabinoid system is involved in a variety of physiological processes, including appetite, pain-sensation, mood, and memory. Cannabinoids can be classified into three different groups: endocannabinoids produced naturally in the body by animals; phytocannabinoids found in cannabis; and synthetic cannabinoids manufactured artificially. Exemplary cannabinoids include, but are not limited to, cannabiodiol (CBD), cannabigerol (CBG), cannibinol (CBN), tetrahydrocannabinoil (THC), and tetrahydrocannabivarin (THCV).

When extracting cannabinoids from cannabis, including from hemp, the extracted cannabinoid can be in the form of a powder or an oil. For example, various methods exist for extracting CBD from cannabis, with some extraction methods producing CBD isolate in the form of a powder, while other extraction methods produce either full-spectrum CBD distillate in the form of an oil, or broad-spectrum CBD distillate in the form of an oil. Currently, full spectrum CBD distillate and broad-spectrum CBD distillate are highly desirable since both products contain additional cannabinoids, terpenes, and other compounds that complete an entourage effect for the consumer. However, because full-spectrum CBD distillate and broad-spectrum CBD distillate are produced in the form of an oil, the primary delivery forms are limited to liquid tinctures, vapor cartridges, and soft-gel capsules. While these delivery forms are effective, they may not always be consumer friendly. Therefore, it would be desirable to provide a free-flowing powder form of full-spectrum or broad-spectrum CBD, as well as other cannabinoids, to the market.

Regulatory issues that exist with respect to certain forms of extracted cannabinoids also create a need for free-flowing powder forms of cannabinoids. For example, the Food and Drug Administration (FDA) currently presents guidance that if an active ingredient possesses FDA approval for a specific indication, then the same active ingredient may not be deployed as a dietary supplement into the mass market. GW Pharmaceuticals has FDA approval on the use of CBD isolate for treatment of epilepsy. The FDA may therefore regulate CBD isolate as a strict active ingredient without any possibility for use as a dietary supplement and/or may regulate CBD isolate in accordance with various dosage amounts. This, in turn, has driven companies towards using CBD distillates (e.g., full spectrum or broad spectrum CBD distillate), since CBD distillate falls outside of the regulations imposed by the FDA on CBD isolate. However, as discussed above, CBD distillates are currently only available in the form of an oil, which can be less user friendly than free flowing powders.

Yet another issue that may arise with respect to cannabinoids in the liquid or powder form is the bio-availability of cannabinoids in a mammal. For a cannabinoid to be effective in providing a benefit to the consumer, there must be good bio-availability of cannabinoid molecule once inside the human system. However, many cannabinoids naturally exhibit low bio-availability. For example, CBD presents a challenge in lacking a significantly high level of bio-absorption in mammal systems.

Previously known efforts to convert cannabinoid oils such as CBD distillate oils to powders have several drawbacks. For example, previously known processes for converting broad spectrum CBD distillate oil into a powder use conventional spray-drying techniques. In these methods, the broad-spectrum CBD distillate oil is first mixed with water, surfactants, and additional solubility enhancers. This mixture is then sprayed in a fine mist in a manner where liquid components evaporate, thereby resulting in a powder. Other known processes for converting CBD distillate oil to a powder use a nano-emulsion method where the CBD distillate oil is emulsified into tiny droplets, and the tiny droplets are then spray-dried to result in a free-flowing powder. However, all of these previously known processes are expensive, time intensive, demand high energy requirements, and/or result in a low active ingredient load. Beyond the costs associated with these known techniques the low active ingredient load causes a limited number of finished product applications to be implemented to the market.

Accordingly, a need exists for improved methods of preparing high bio-availability free-flowing powder from cannabinoid oils that address some or all of the issues identified above.

### BRIEF DESCRIPTION OF THE DRAWINGS

FIG. 1 is a flow chart illustrating a process for preparing free-flowing powder from cannabinoid oil (e.g., CBD distillate oil) according to various embodiments described herein.

### DETAILED DESCRIPTION

The methods described herein are applicable to any and all cannabinoid oils. However, for the sake of simplicity, this Detailed Description primarily discusses the conversion of CBD distillate oil to a free-flowing powder. Regardless of this focus, this disclosure should not be construed or interpreted as being limited to the conversion of only CBD distillate oil to free-flowing powder, since the same methods described herein can be used to convert any cannabinoid oil to a free-flowing powder.

As used herein, the term “CBD distillate oil” and variations thereof will refer to both broad spectrum CBD distillate oil and full spectrum CBD distillate oil. The methods described herein are equally applicable to broad spectrum CBD distillate oil and full spectrum CBD distillate oil as it relates to converting these oils to free-flowing powders.

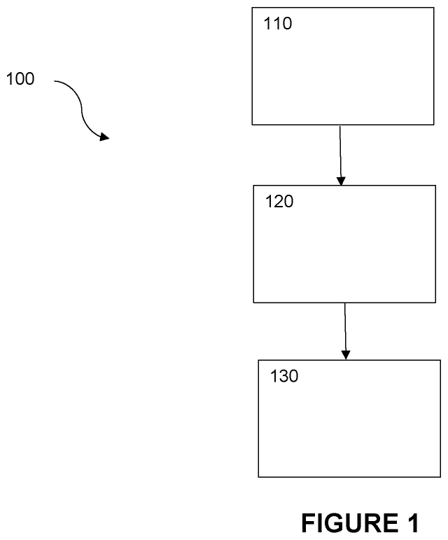

With reference to FIG. 1, a method 100 for producing free flowing powder from CBD distillate oil generally comprises a step 110 of granulating the CBD distillate oil using a granulating agent, a step 120 of size adjusting the granules, and a step of 130 of adding flow agent to the granules.

In step 110, a granulation process is carried out in which CBD distillate oil is mixed with one or more granulating agents to form initial granules. Any suitable granulating apparatus can be used in order to carry out the granulation step 110. Either low or high shear granulation processes can be used. In some embodiments, the granulation apparatus (i.e., granulator) is charged with the granulating agent. The granulating agent can be a cellulose of mineral derivative provided in a powder form. Exemplary though non-limiting granulation agents in various derivatives, grades, and preparation forms include microcrystalline cellulose, ethyl cellulose, hydroxypropyl methyl cellulose, and dicalcium phosphate. One or more granulating agents can be used, and the quantity of granulating agent charged into the granulator is not limited. The amount of granulating agent used in step 110 will generally depend on the amount of CBD distillate oil that will be added in the granulation step, as well as other considerations, such as the desired characteristics of the final powder, the size/capacity of the granulation equipment, etc.

Once the granulator is charged with granulation agent, mixing at, e.g., medium speed can be initiated. At this point, CBD distillate oil is metered into the granulator to be mixed with the granulation agent and begin the process of forming granules. In some embodiments, the CBD distillate oil is warmed prior to being metered into the granulator. For example, the CBD distillate oil can be warmed to a temperature in the range of about 55 to about 95 deg. C. Warming the CBD distillate oil in this manner can facilitate dispersion. In some embodiments, the CBD distillate oil is metered into the granulator in a quantitative method for systematic addition of CBD distillate oil to the granulator. The specific rate at which the CBD distillate oil is metered into the granulator is generally not limited, and can vary based on, e.g., batch size. In some embodiments, the CBD distillate oil is added at a rate of from about 5 to about 50 grams per minute.

Once the CBD distillate oil is added to the granulator, the CBD distillate oil and granulating agent are sufficiently mixed until a homogenous mixture comprising granules is formed. In some embodiments, the homogenous mixture is allowed to cool prior to the next step. For example, the mixture can be allowed to cool to a temperature below 40 deg. C.

Other granulation techniques can also be used for step 110. For example, fluid bed hot melt granulation can also be used to mix together CBD distillate oil and a granulation agent to form granules. In fluid bed hot melt granulation, CBD distillate oil is warmed to a temperature in the range of from about 55 to about 95 deg. C. and then sprayed onto a carrier in a fluid bed. The rate of spray can be, e.g., between 10 and 80 grams per minute. The granulation mixture formed by this method can then be allowed to cool to, e.g., less than 40 deg. C.

In still other granulation methods, a hot melt system in which CBD distillate oil is sprayed on to a carrier can also be used. The carrier can be, e.g., a polysaccharide microcrystalline cellulose micro-sphere. In a fluid bed, CBD distillate oil is warmed to a temperature in the range of from about 55 to about 95 deg. C. and then sprayed on the carrier (e.g., polysaccharide microcrystalline cellulose micro-sphere) in a fluid bed. The rate of spray can be, e.g., between 10 and 80 grams per minute. The granulation mixture formed by this method can then be allowed to cool to, e.g., less than 40 deg. C.

In step 120, the size of the granules formed in step 110 is adjusted. In some embodiments, size adjustment is carried out by adding additional binder and/or fillers to the granules formed in step 110 in order to increase the size of the granules. Any suitable binders and/or fillers can be used in any desired amount. In some embodiments, the binders/fillers are cellulose or mineral derivatives, such as microcrystalline cellulose, hydroxypropyl methylcellulose, and/or dicalcium phosphate.

Other components can also be added to the granule mixture at step 120 to adjust the size of the granules and/or provide other desired characteristics to the granules. For example, in some embodiments, bioavailability enhancers (also referred to as molecular inhibitors) and/or time-release components can be added. Exemplary though non-limiting bioavailability enhancers include flavonoids, alkaloids, chitosan, curcum in, cyclocsporine, diosmin, emodin, gallic acid, genistein, grapefruit, citrus fruit extracts, lycopene, lysergol, Moringa oleifera pods, naringin, peppermint oil, quercetin, resveratrol, sinomenine, sodium caprate, and sodium cholate. Bioavailability enhancers help active ingredients such as cannabinoids in the powder described herein pass through the liver without being metabolized and/or degraded. Liver enzymes have a higher affinity for the bioavailability enhancers, and thus the bioavailability enhancers lock up the liver enzymes while the cannabinoid passes through the liver without degradation. Exemplary though non-limiting time release components include ethyl cellulose, eudragit, hydroxypropyl methylcellulose, hydrogenated cottonseed oil, shellac, etc. These components can be added or removed based on the desired characteristics of the final product.

In step 130, after final homogenous mixing of all components added in steps 110 and 120 to form appropriately sized granules and after sufficient cooling of the mixture, a flow agent can be added to the granulated mixture to further enhance the free-flowing nature of the produced powder. Exemplary, though non-limiting flow agents include talc, corn starch, etc. These flow agents (in any combination) can be added in any quantity needed to achieve desired final results with respect to flowability. In some embodiments, the flow agent is added to the granulated mixture using a low shear blender, though other techniques and/or equipment can be used.

### Example

Table 1 provides an ingredients and quantity range list for exemplary finished powders produced using the methods described herein.

TABLE 1

| Material | Range wt % |
|---|---|
| CBD distillate oil | 20-60 |
| Cellulose Derivative | 0-60 |
| Calcium | 0-60 |
| Enteric Polymers | 0-25 |
| Bioavailability Enhancers | 0-50 |
| Flow agent | 0-2 |

From the foregoing, it will be appreciated that specific embodiments of the invention have been described herein for purposes of illustration, but that various modifications may be made without deviating from the scope of the invention. Accordingly, the invention is not limited except as by the appended claims.

1. A method for producing free flowing powder from a cannabinoid oil, the method comprising:
granulating a cannabinoid oil with at least one granulating agent to thereby produce a granulated mixture;
adjusting the size of granules in the granulated mixture by mixing one or more binders or fillers with the granulated mixture; and
mixing at least one flow agent with the granulated mixture to form a free-flowing powder.

2. The method of claim 1, wherein the cannabinoid oil is cannabidiol distillate oil.

3. The method of claim 1, wherein the cannabinoid oil is broad spectrum cannabidiol oil.

4. The method of claim 1, wherein the cannabinoid oil is full spectrum cannabidiol oil.

5. The method of claim 1, wherein the at least one granulating agent comprises at least one of microcrystalline cellulose, ethyl cellulose, hydroxypropyl methylcellulose, and dicalcium phosphate.

6. The method of claim 1, wherein granulating the cannabinoid oil with the at least one granulating agent comprises:
loading the at least one granulating agent in a granulator;
mixing the at least one granulating agent inside the granulator; and
while mixing continues, metering the cannabinoid oil into the granulator.

7. The method of claim 6, wherein the cannabinoid oil is heated to a temperature in the range of from about 55 to about 95 deg. C. prior to being metered into the granulator.

8. The method of claim 7, wherein metering the cannabinoid oil into the granulator comprises spraying the cannabinoid oil into the granulator.

9. The method of claim 1, wherein the granulated mixture is cooled to a temperature below about 40 deg. C. prior to mixing the one or more binders or fillers to the granulated mixture.

10. The method of claim 1, wherein the one or more binders or fillers comprises at least one of microcrystalline cellulose, hydroxypropyl methylcellulose, and dicalcium phosphate.

11. The method of claim 1, further comprising:
mixing at least one bioavailability enhancer with the granulated mixture prior to mixing the at least one flow agent with the granulated mixture.

12. The method of claim 11, wherein the at least one bioavailability enhancer comprises at least one of flavonoids, alkaloids, chitosan, curcumin, cyclosporine, diosmin, emodin, gallic acid, genistein, grapefruit, citrus fruit extracts, lycopene, lysergol, Moringa oleifera pods, naringin, peppermint oil, quercetin, resveratrol, sinomenine, sodium caprate, and sodium cholate.

13. The method of claim 1, further comprising mixing at least one time release component with the granulated mixture prior to mixing the at least one flow agent with the granulated mixture.

14. The method of claim 13, wherein the at least one time release component comprises at least one of ethyl cellulose, methacrylic acid-methyl acrylate copolymer, hydroxypropyl methylcellulose, hydrogenated cottonseed oil, and shellac.

15. The method of claim 1, wherein the at least one flow agent comprises at least one of talc and corn starch.
